(19) United States
(12) Patent Application Publication
(10) Pub. No.: US 2026/0325032 A1
(43) Pub. Date: Oct. 22, 2026

(54) ELECTRODE FOR ROBOTIC SURGERY

(71) Applicant: Senzime AB (publ.), Uppsala (SE)

(72) Inventors: Anders JAKOBSON, Uppsala (SE); Sorin BRULL, Uppsala (SE)

(73) Assignee: Senzime AB (publ.), Uppsala (SE)

(21) Appl. No.: 19/183,014

(22) Filed: Apr. 18, 2025

(51) Int. Cl. A61B 5/294; A61B 5/00; A61B 5/388

(52) U.S. Cl. CPC A61B 5/294; A61B 5/388; A61B 5/4848; A61B 2562/043; A61B 2562/164

(57) ABSTRACT

An electrode system configured to obtain neuromuscular block signals for a neuromuscular block monitor, the electrode system comprising: a flexible substrate having an elongated shape, wherein the substrate comprises a connector interface arranged at a first end of the electrode system, wherein the substrate comprises a plurality of stimuli electrodes arranged at a second opposite end of the electrode system, wherein the substrate further comprises wherein the substrate comprises a plurality of response electrodes arranged between the first and second ends of the electrode system, wherein the substrate further comprises a plurality of conductive traces carried on the flexible substrate, wherein the plurality of conductive traces further comprises conductive traces that routes from a respective response electrode, to the connector interface wherein the plurality of conductive traces further comprises conductive traces that routes from a respective stimulus electrode to the connector interface.

### TECHNICAL FIELD

The present disclosure relates to electrode systems for anesthetizing monitoring systems capable of measuring a level of neuromuscular block of a subject.

### BACKGROUND

In hospitals around the world, patients are given muscle relaxants also called neuromuscular blocking agents, NMBAs, which inhibit neuromuscular transmission. These relaxant agents decrease muscle tension and suppress reflex contractions. The different types of NMBAs have different onset and durations but normally they have to be administered repeatedly, and the dose needs to be titrated appropriately, to maintain a desired tissue (neuromuscular junction) concentration level or blood concentration throughout a surgical procedure.

Neuromuscular monitoring systems using evoked electromyography, EMG, is nowadays regarded as the new gold standard for measuring the degree of neuromuscular function or, equivalently, the depth of muscular block. Evoked EMG involves sending a stimulus signal to stimulating electrodes positioned on the patient's body (a peripheral nerve) and monitoring (recording) the response signal from receiving electrodes also positioned on the patient's body (more precisely, positioned on the muscle innervated by the particular stimulated nerve).

The electrodes are typically coupled via an interface to the neuromuscular monitoring systems. A problem is that the location of the interface between the electrode connector and the connecting cable is often blocked by other equipment when the patient is prepared for surgery.

Thus, there is a need for an improved electrode system for monitoring neuromuscular function during anesthesia and surgery.

### OBJECTS OF THE INVENTION

An objective of embodiments of the present invention is to provide a solution which mitigates or solves the drawbacks and problems described above.

### SUMMARY OF THE INVENTION

The above and further objectives are achieved by the subject matter described herein. Further advantageous implementation forms of the invention are described herein. The invention is set out in the appended claims. The scope of the invention is defined by the claims which are incorporated into this section by reference.

According to a first aspect of the invention the object of the invention is achieved by an electrode system configured to obtain neuromuscular block signals for a neuromuscular block monitor, the electrode system comprising: a flexible substrate having an elongated shape, wherein the substrate comprises a connector interface arranged at a first end of the electrode system, wherein the substrate comprises a plurality of stimuli electrodes arranged at a second opposite end of the electrode system, wherein the substrate further comprises a plurality of response electrodes arranged between the first and second ends of the electrode system, wherein the substrate further comprises a plurality of conductive traces carried on the flexible substrate, wherein the plurality of conductive traces comprises conductive traces that routes from a respective response electrode to the connector interface wherein the plurality of conductive traces further comprises conductive traces that routes from a respective stimulus electrode to the connector interface.

In one embodiment, the substrate further comprises a plurality of slits forming interconnected parts of the substrate.

In one embodiment, the interconnected parts of the substrate comprises a selection from the plurality of stimuli electrodes, the plurality of response electrodes and segments of the plurality of conductive traces.

In one embodiment, the interconnected parts of the substrate comprise a first part where a plurality of stimuli electrodes are arranged, a second and third part forming a first adaptive shape, a fourth part where a first response electrode is arranged, a fifth and sixth part forming a second adaptive shape and a seventh part where a second response electrode is arranged.

In one embodiment, the substrate further comprises a plurality of islands and one or more bridges between the plurality of islands and the connector interface.

In one embodiment, a first island comprises the plurality of stimuli electrodes, a second island comprises at least one of the response electrodes, and a third island comprises the connector interface.

In one embodiment, the second island comprises a first response electrode, and the third island comprises a second response electrode.

In one embodiment, a first bridge is configured to connect the first island to the second island and a second bridge is configured to connect the second island to the third island.

In one embodiment, the first bridge is provided with an adaptive shape.

In one embodiment, the electrode system further comprises a second connector interface arranged at the second end of the electrode system, wherein the plurality of conductive traces further routes to the second connector interface.

In one embodiment, geometric centers of the stimuli electrodes are arranged with an offset from a longitudinal axis of the substrate.

In one embodiment, a first group of stimuli electrodes is arranged with a first offset from the longitudinal axis and a second group of stimuli electrode arranged with a second offset from the longitudinal axis on an opposite side of the longitudinal axis in relation to the first group of stimuli electrodes.

In one embodiment, the first offset and the second offset are identical.

In one embodiment, geometric centers of the respective stimuli electrodes are centered along a second axis arranged perpendicular to the longitudinal axis.

Further applications and advantages of embodiments of the invention will be apparent from the following detailed description.

### BRIEF DESCRIPTION OF THE DRAWINGS

FIG. 1 shows an electrode system according to one or more embodiments of the present disclosure.

FIG. 2 shows an electrode system provided with slits according to one or more embodiments of the present disclosure.

FIG. 3 shows an example of electrode system provided with islands and bridges according to one or more embodiments of the present disclosure.

FIG. 4A-B shows examples of the electrode system with an offset between stimuli electrodes according to one or more embodiments of the present disclosure.

FIG. 5 shows an example of electrode system with electrodes mirrored on either side of the longitudinal axis according to one or more embodiments of the present disclosure.

FIG. 6 shows an example of a neuromuscular block monitoring system configured to monitor neuromuscular block according to one or more embodiments of the present disclosure.

### DETAILED DESCRIPTION

Stimulating muscles to generate twitches has many applications, from physical training to advanced therapy.

Electromyography (EMG) is a technique for evaluating and recording the electrical activity produced by muscle in response to stimulation of its nerve, and is used in many professions e.g., by physiotherapists, kinesiologists and biomedical engineers. An apparatus, such as an electromyograph, detects the electric potential generated by muscle cells when these cells are electrically or neurologically activated.

In anesthesia, neuromuscular blocking agents may be administered typically to facilitate surgery. When neuromuscular blocking agents are administered, it is desirable to monitor neuromuscular function of the patient using a neuromuscular block monitor. This typically involves the electrical stimulation of a motor nerve and monitoring the response of the muscle supplied by that nerve and may be used from the initial administration of neuromuscular blocking agents to when the patient is recovered from the neuromuscular blockade.

In this application, a Train-of-Four (TOF) stimulation signal may typically be sent to a nerve, and the number of responses will be analyzed. The pulses may e.g., be analyzed with regards to TOF count (count of pulses) or TOF ratio (amplitude ratio between the fourth, T4, and first, T1, received pulse—or T4/T1; TOF ratio; TOFR). As the neuromuscular block also affects breathing functions of the patient, accuracy of the monitoring is of great importance. In other words, the application in anaesthesia differs from the other applications mentioned above in that the accuracy of quantification (e.g., shape, amplitude, area, duration, etc.) is of great importance; therefore, the signal quality of received signals is also of great importance.

An important factor in the accuracy of the monitoring is therefore related to the quality of the signal.

A problem with existing electrode systems is that interference is picked up by conductors between an interface of the electrode system and the response electrodes, that are recording the response signal resulting from the stimulus signal. In other words, the signal is degraded by interference of such as noise.

A further problem with existing electrode systems is that the location of the electrode interface occupies a footprint in areas where other equipment competes for space, e.g., close to the distal forearm and hand where other equipment such as a venous catheter may be located.

A further problem is that when a patient is prepared for surgery, the location of the interface may be covered by bed linen or other clothing, making it difficult to connect the monitor.

The present disclosure solves these problems by relocating the interface, thereby making it more accessible whilst simultaneously shortening conductors, such as traces on a substrate, and thereby reducing the ability to pick up interference.

An “or” in this description and the corresponding claims is to be understood as a mathematical OR which covers “and” and “or”, and is not to be understood as an XOR (exclusive OR). The indefinite article “a” in this disclosure and claims is not limited to “one” and can also be understood as “one or more”, i.e., plural.

In the present disclosure, the term “stimuli points” denotes points on a patient's body where the best possible transfer of a stimulus signal to a nerve can be made.

In the present disclosure, the term “response points” denotes points on a patient's body where the best possible transfer of a response signal from a muscle can be made.

In the present disclosure, the term “electrode system” denotes a system that comprises all technical features to obtain neuromuscular block signals by a neuromuscular block monitor. This includes stimuli electrodes to be placed on stimuli points, response electrodes to be placed on response points, an interface to electrically and communicatively couple the stimuli electrodes and response electrodes to a neuromuscular block monitor, and conductive traces arranged on a substrate and configured to communicatively couple the stimuli electrodes and response electrodes to the interface.

In the present disclosure, the term “adaptive shape” denotes a shape of a substrate that allows the substrate to stretch, unwind, unfold, uncurl, extend, etc. In the present disclosure, the term “substrate” denotes a flexible substrate capable of adapting to the surface of a patient's body and to hold the conductive traces, the stimuli electrodes and the response electrodes.

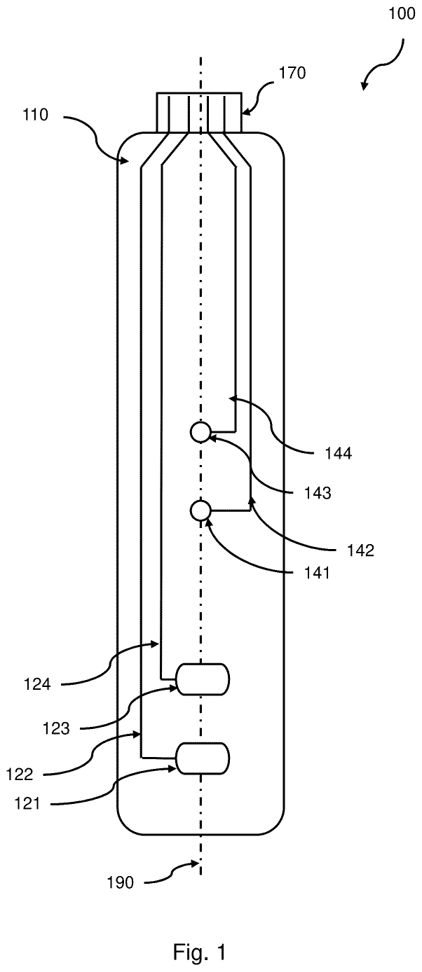

FIG. 1 shows an electrode system 100 according to one or more embodiments of the present disclosure. The electrode system 100 is configured to obtain neuromuscular block signals for a neuromuscular block monitor. The electrode system 100 comprising:

A flexible substrate 110 having an elongated shape. The exact shape of the substrate 110 depends on the application, and is designed to conform with stimuli points and response points on a patient's body.

The substrate 110 further comprises a connector interface 170 arranged at a first end of the electrode system. The purpose of the connector interface 170 is to communicatively and electrically couple the electrode system to the neuromuscular block monitor, typically by connecting to a connector.

The substrate 110 further comprises a plurality of stimuli electrodes 121, 123 arranged at a second opposite end of the electrode system. The stimuli electrodes are typically elongated and oriented with a longitudinal axis perpendicular to a longitudinal axis of the elongated electrode system 100. An example of such electrodes may be found in EP2956052B1. The stimuli electrodes 121, 123 are shown as an elongated shape with rounded corners in FIG. 1, but it understood that any suitable shape may be used without departing from the present disclosure, e.g., round, semi-circular or other geometric shapes (half-moon, diamond, etc.).

The substrate 110 further comprises a plurality of response electrodes 141, 143 arranged between the first and second ends of the electrode system 100. The response electrodes 141, 143 are shown as circular in FIG. 1, but it understood that any suitable shape may be used without departing from the present disclosure.

The substrate 110 further comprises a plurality of conductive traces 122, 124, 142, 144 carried on the flexible substrate. The plurality of conductive traces comprises conductive traces 142, 144 that routes from a respective response electrode 141, 143 to the connector interface 170. The plurality of conductive traces further comprises conductive traces 122,124 that routes from a respective stimulus electrode 121, 123 to the connector interface 170.

One advantage may be that in certain surgeries, the arm containing the electrode may be wrapped in surgical padding and/or surgical drapes. This electrode connector may then be left outside the wrapping, such that connection to the connecting cable is facilitated. Second, since the electrode connector to cable connector connection is outside of the wrapped arm, any disconnection or poor connection can be readily identified and corrected. Third, in surgical cases in which the operating table it turned away from the anesthesiologist space and the anesthesia workstation such that the patient's head is distant from the anesthesiologist's workspace, the electrode connector to cable connector connection is in a straight line to the anesthesiologist, facilitating access to this connection in order to troubleshoot any potential misconnections or disconnections. Fourth, this electrode connector orientation (in a straight line away from the hand) allows the electrode connection to the cable connection to be away from the actual electrode (instead of being folded over the existing electrodes), thereby minimizing potential interference with the electrode signal acquisition.

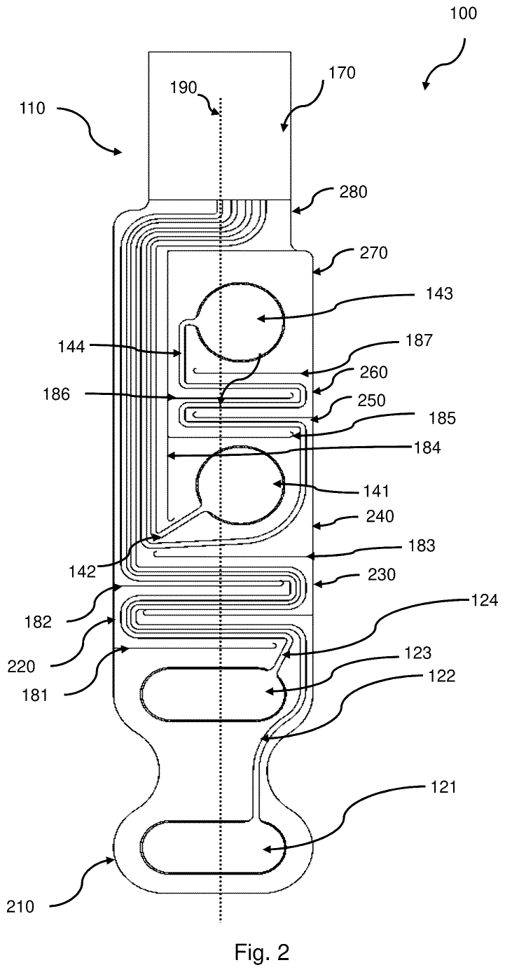

FIG. 2 shows an electrode system 100 provided with slits according to one or more embodiments of the present disclosure. In this embodiment, the substrate 110 further comprises a plurality of slits 181-187 forming interconnected parts of the substrate 110.

Optionally, the substrate 110 is further provided with bays cutting in between the plurality of stimuli electrodes 121, 123. In other words, the outer edge of the substrate 110, between the stimuli electrodes 121, 123, extend towards the longitudinal axis 190 of the substrate 110, e.g., as a curve or semicircle.

The electrode system shown in FIG. 2 includes all features described in relation to FIG. 1.

As mentioned above, the slits 181-187 form interconnected parts of the substrate 110, where each of the interconnected parts comprises a selection from the plurality of stimuli electrodes 121, 123, the plurality of response electrodes (141, 143) and segments of the plurality of conductive traces (122, 123, 142, 144).

In other words, the slits 181-187 provides the ability to spatially separate stimuli electrodes and/or response electrodes to better conform to stimuli points and response points on a patient's body.

In the example shown in FIG. 2, the interconnected parts of the substrate 110 comprise a part 210 where the plurality of stimuli electrodes 121, 123 are arranged. Optionally, the side edge of the part 210 of the substrate 110 is arranged with bays that extend in from the side towards the longitudinal axis. The interconnected parts further comprise a part 220 and part 230 forming a first adaptive shape, allowing variation of distances between the stimuli electrodes 121, 123 and/or the response electrodes 141, 143.

The interconnected parts of the substrate 110 further comprises a part 240, where a response electrode 141 is arranged.

The interconnected parts further comprise a part 250 and a part 260 forming a second adaptive shape, allowing variation/adaption of distances between the response electrodes 141, 143. This assures that response electrodes better conform to stimuli points and response points on a patient's body.

The interconnected parts further comprise a part 270 where the response electrode 143 is arranged.

The interconnected parts further comprise a part 280 where the connector interface 170 is arranged.

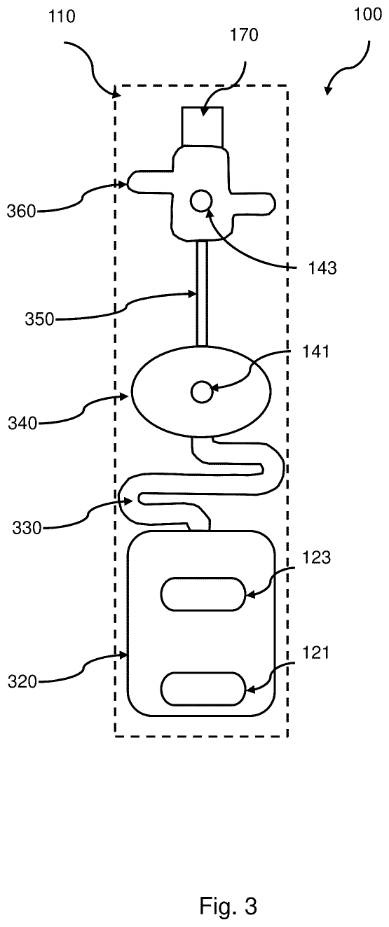

FIG. 3 shows an example of electrode system 100 provided with islands and bridges according to one or more embodiments of the present disclosure. In this embodiment, the electrode system 100 further comprises a plurality of islands 320, 340, 360, and one or more bridges 330, 350. The bridges 330, 350, are arranged between the plurality of islands and the connector interface 170.

In this example, the elongated shape of the substrate 110 is indicated by the dashed box.

The islands comprises a selection from the plurality of stimuli electrodes 121, 123, the plurality of response electrodes 141, 143 and segments of the plurality of conductive traces 122, 123, 142, 144 described in relation to FIG. 2.

The one or more bridges 330, 350 comprise segments of the plurality of conductive traces 122, 123, 142, 144 described in relation to FIG. 2.

The plurality of islands 320, 340, 360, and the one or more bridges 330, 350 substantially map to the interconnected parts of the substrate 110 shown in relation to FIG. 2.

The substrate 110 of the electrode system 100 comprises an island 320. The island 320 comprising the plurality of stimuli electrodes 121, 123. Optionally, the plurality of stimuli electrodes 121, 123 may be distributed on multiple islands linked by bridges (not shown) depending on application. E.g., one stimulus electrode on each island. Separating the plurality of stimuli electrodes 121, 123 may in some applications be required to better adapt to locations of stimuli points and response points on a patient's body.

In other words, to better adapt to stimuli points and/or response points where the best possible transfer of a stimulus signal to a nerve can be made or the best possible transfer of a response signal from a muscle can be made.

The substrate 110 of the electrode system 100 further comprises yet an island 340. The island 340 comprises at least one of the response electrodes 141.

The substrate 110 of the electrode system 100 further comprises yet an island 360. The island 360 comprises the connector interface 170.

Optionally, the island 360 also comprises the response electrode 143. In other words, the connector interface 170 and the response electrode 143 are arranged on the same island.

Alternatively, the substrate 110 of the electrode system 100 further comprises yet an island (not shown), that comprises the response electrode 143. I.e., a separate island for the second response electrode 143 that is linked by bridges to islands 340 and 360. In other words, the connector interface 170 and the second response electrode 143 are arranged on separate islands and linked by bridges.

Alternatively, the island 340 comprises the second response electrode 143 (not shown). In other words, the response electrodes 141 and 43 are arranged on the same island.

Separating the plurality of stimuli electrodes 121, 123 on different islands or arranging them together on the same island may in some applications be required to better adapt to locations of stimuli points on a patient's body.

The one or more bridges 330, 350 are configured or arranged to mechanically link the islands and to provide a path for the plurality of conductive traces 122, 123, 142, 144.

The substrate 110 of the electrode system 100 further comprises a bridge 330 that is configured to connect or couple the island 320 to the island 340. In one embodiment, the bridge 130 is provided with an adaptive shape that allows a distance between the island 320 to the island 340 to be adapted. In other words, the bridge is configured or formed in an adaptive shape that allows the substrate to stretch, unwind, unfold, uncurl, extend, etc. The adaptive shape may be in the form of a serpentine shape or a zig-zag shape. As mentioned, this allows for better adaptation to locations of stimuli points and/or response points on a patient's body.

The substrate 110 of the electrode system 100 further comprises a bridge 350 that is configured to connect the island 340 to the island 360. In one embodiment, the bridge 350 is provided with a stretchable shape that allows a distance between the island 340 to the island 360 to be adapted. As mentioned, this allows better adaptation to locations of stimuli points and/or response points on a patient's body.

In alternative embodiments, any of the electrode systems described in relation to FIG. 2 or FIG. 3 comprises an additional connector interface 171 (not shown). The second connector interface 171 is arranged at the second end of the electrode system, wherein the plurality of conductive traces further route to the additional connector interface 171. In other words, the additional connector interface 171 is communicatively or electrically coupled to the stimuli electrodes 121, 123 and the response electrodes 141 in the same manner as the connector interface 170, such that the neuromuscular block monitor can connect.

In applications where the arrangement of stimuli points and/or response points on a patient's body is well known and remains substantially fixed, it may be advantageous to relatively displace the stimuli electrodes. An advantage is that monitoring of responses can still occur even if the stimulation point/site is relatively remote from the response point/site.

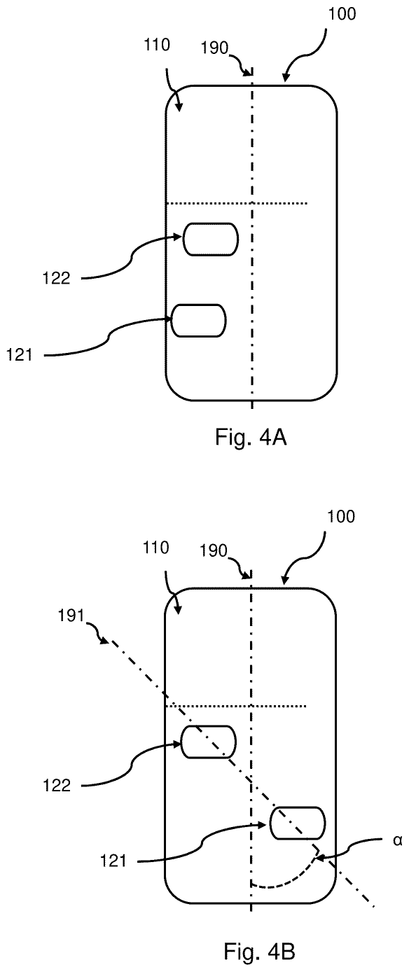

FIG. 4A shows an example of the electrode system 100 with an offset electrodes according to one or more embodiments of the present disclosure.

In one embodiment, a geometric center of the stimuli electrodes 121, 123 are arranged with an offset from a longitudinal axis 190 of the substrate 110.

In this context, “geometric center” refers to a center point of the electrodes, and this center point is offset from the longitudinal axis 190, as illustrated in FIG. 4A.

In one embodiment, a first group of stimuli electrodes 121 is arranged with an offset from the longitudinal axis 190 and a second group of stimulus electrode 122 is arranged with the same offset from the longitudinal axis 190 on the same side of the longitudinal axis (190) in relation to the first group of stimulus electrodes 121. In other words, the first group and second group is offset with a distance from and parallel to the longitudinal axis 190

In one embodiment (not shown), the first group of stimuli electrodes 121 is arranged with a first offset from the longitudinal axis 190 and the second group of stimuli electrode 122 is arranged with a second offset from the longitudinal axis 190 on the opposite side of the longitudinal axis (190) in relation to the first group of stimuli electrodes 121. In other words, the first group is offset with a first distance to one side of the substrate 110. The second group is offset with a second distance to the opposite side of the longitudinal axis 190 in relation to the first group of stimuli electrodes 121. i.e., the groups may be symmetrically offset from longitudinal axis 190 or asymmetrically offset from longitudinal axis 190.

In one embodiment, the first offset and the second offset are identical. I.e., the offsets are symmetrical. In other words, the first second group of electrodes are arranged to be mirrored on either side of the longitudinal axis 190.

FIG. 4B shows a further example of the electrode system 100 with stimuli electrodes arranged at an angle to the longitudinal axis 190 according to one or more embodiments of the present disclosure.

In this embodiment, geometric center(s) of the first group of stimuli electrodes 121 and geometric center(s) of the second group of stimuli electrodes 122 are arranged along an axis 192. The axis 192 being arranged at an angle a to the longitudinal axis 190. The angle may e.g., be selected as 30 or 45 degrees or any other suitable angle.

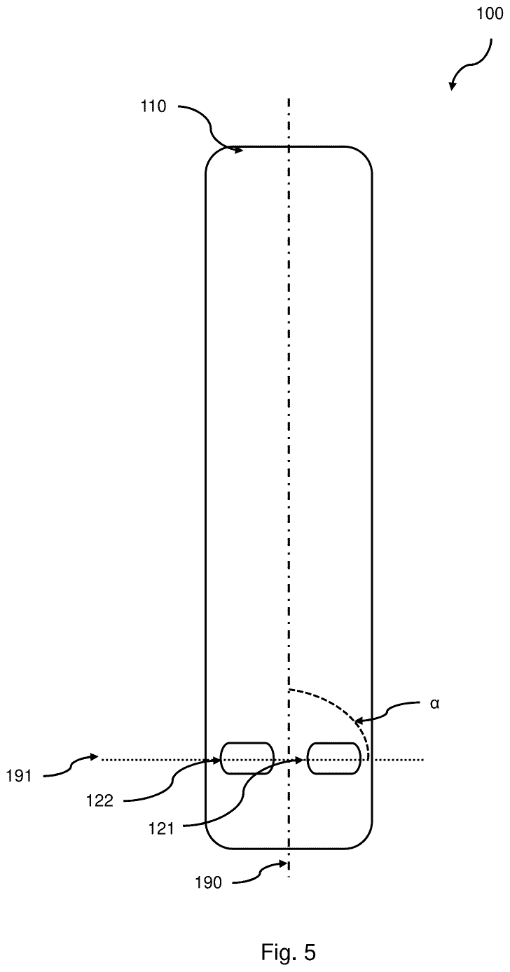

FIG. 5 shows an example of electrode system 100 with electrodes mirrored on either side of the longitudinal axis 190 according to one or more embodiments of the present disclosure.

In one embodiment, geometric centers of the respective stimuli electrodes 121, 123 are also centered along an axis 192. The axis 192 being arranged perpendicular to the longitudinal axis 190. In other words, the axis 192 being arranged at an angle a to the longitudinal axis 190. The angle a may e.g., be selected to 90, or other degrees.

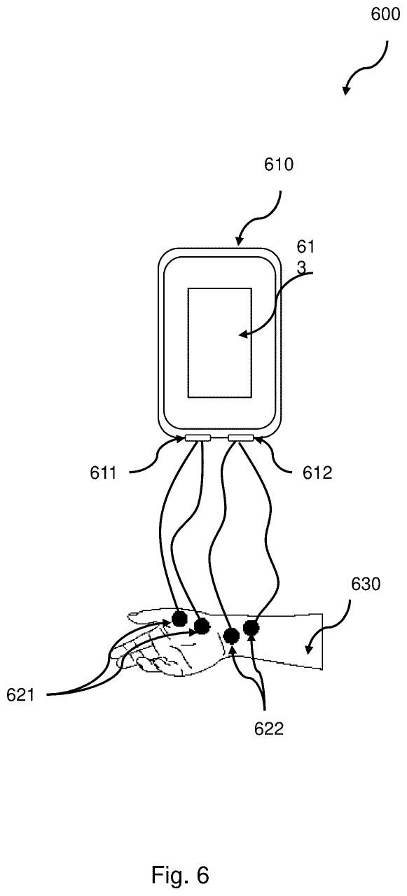

FIG. 6 shows an example of a neuromuscular block monitoring system 600 configured to monitor neuromuscular block according to one or more embodiments of the present disclosure. The neuromuscular block monitoring system 600 may comprise a neuromuscular block monitoring unit 610 provided with an input port 611 and an output port 612 according to embodiments described herein. The neuromuscular block monitoring system 600 may further comprise stimulating electrodes 622 configured to be electrically couplable to the output port 612 and being configured to receive a stimulus signal from the output port 612 and deliver the stimulus signal to the anesthetized patient 630. The neuromuscular block monitoring system 600 may further comprise receiving electrodes 621 configured to be electrically couplable to the input port 611 and being configured to obtain a response signal in response to the stimulus signal obtained from the anesthetized patient 630.

The receiving electrodes 621 and stimulating electrodes 622 may be arranged on the electrode system described herein.

Finally, it should be understood that the invention is not limited to the embodiments described above, but also relates to and incorporates all embodiments within the scope of the appended independent claims.

1. An electrode system configured to obtain neuromuscular block signals for a neuromuscular block monitor, the electrode system comprising:
a flexible substrate having an elongated shape,
wherein the substrate comprises a connector interface arranged at a first end of the electrode system,
wherein the substrate comprises a plurality of stimuli electrodes arranged at a second opposite end of the electrode system,
wherein the substrate further comprises wherein the substrate comprises a plurality of response electrodes arranged between the first and second ends of the electrode system,
wherein the substrate further comprises a plurality of conductive traces carried on the flexible substrate, wherein the plurality of conductive traces further comprises conductive traces that routes from a respective response electrode to the connector interface wherein the plurality of conductive traces further comprises conductive traces that routes from a respective stimulus electrode to the connector interface.

2. The electrode system according to claim 1, wherein the substrate further comprises a plurality of slits forming interconnected parts of the substrate.

3. The electrode system according to claim 2 wherein the interconnected parts of the substrate comprises a selection from the plurality of stimuli electrodes, the plurality of response electrodes and segments of the plurality of conductive traces.

4. The electrode system according to claim 3 wherein the interconnected parts of the substrate comprises a first part where the a plurality of stimuli electrodes are arranged, a second and third part forming a first adaptive shape, a fourth part where a first response electrode is arranged, a fifth and sixth part forming a second adaptive shape and a seventh part where a second response electrode is arranged.

5. The electrode system according claim 1, wherein the substrate further comprises a plurality of islands and one or more bridges between the plurality of islands and the connector interface.

6. The electrode system according to claim 5, wherein a first island comprises the plurality of stimuli electrodes, a second island comprises at least one of the response electrodes, and a third island comprises the connector interface.

7. The electrode system according to claim 6, wherein the second island comprises a first response electrode, and the third island comprises a second response electrode.

8. The electrode system according to claim 6, wherein a first bridge is configured to connect the first island to the second island and a second bridge is configured to connect the second island to the third island.

9. The electrode system according to claim 8, wherein the first bridge is provided with an adaptive shape.

10. The electrode system according to claim 1, further comprising a second connector interface arranged at the second end of the electrode system, wherein the plurality of conductive traces further routes to the second connector interface.

11. The electrode system according to claim 1, wherein a geometric center of the stimuli electrode/s are arranged with an offset from a longitudinal axis of the substrate.

12. The electrode system according to claim 11, further wherein a first group of stimuli electrodes is arranged with a first offset from the longitudinal axis and a second group of stimuli electrode is arranged with a second offset from the longitudinal axis on an opposite side of the longitudinal axis in relation to the first group of stimuli electrodes.

13. The electrode system according to claim 12, wherein the first offset and the second offset are identical.

14. The electrode system according to claim 13, wherein geometric centers of the respective stimuli electrodes are centered along a second axis arranged perpendicular to the longitudinal axis.
